(19) United States
(12) Patent Application Publication
(10) Pub. No.: US 2024/0000654 A1
(43) Pub. Date: Jan. 4, 2024

(54) PRESSURE MODULE AND THERMOTHERAPY APPARATUS COMPRISING SAME

(71) Applicant: CERAGEM CO., LTD., Cheonan-si, Chungcheongnam-do (KR)

(72) Inventors: Ki Sung Kim, Cheonan-si, Chungcheongnam-do (KR); Yong Hee Kim, Cheonan-si, Chungcheongnam-do (KR); Jae Hwa Kim, Cheonan-si, Chungcheongnam-do (KR); Dong Myoung Lee, Asan-si, Chungcheongnam-do (KR)

(21) Appl. No.: 18/254,831

(22) PCT Filed: Oct. 18, 2021
(86) PCT No.: PCT/KR2021/014451

§ 371 Date: May 26, 2023

(30) Foreign Application Priority Data
Nov. 30, 2020 (KR) 10-2020-0163877

(51) Int. Cl. A61H 15/02; A61H 15/00

(52) U.S. Cl. CPC A61H 15/02; A61H 15/0078; A61H 2203/0443; A61H 2201/0207; A61H 2201/0228

(57) ABSTRACT

The pressure module according to an embodiment of the present invention, which is for thermotherapy, may comprise: a pressure unit which has an interior space; a heat-generating unit which is inserted in the interior space and generates heat so that the pressure unit is heated; and an electromagnetic induction unit which generates an induced current so that the heat-generating unit generates heat.

### TECHNICAL FIELD

The present invention relates to a pressure module and a thermotherapy apparatus including the same. More specifically, the present invention relates to a pressure module for thermotherapy and a thermotherapy apparatus including the same.

### BACKGROUND ART

Conventionally, in order to relieve acute or chronic pain in muscle and nervous tissues of a vertebral part generated due to long, continuous work in an improper posture and habituation of the inappropriate posture, to improve blood circulation of a body, and to relieve instantaneous muscle stitch, a thermotherapy apparatus has been widely used to improve blood circulation by providing a heat stimulus to a pain generation site while moving along a body part.

The conventional thermotherapy apparatus used for such thermotherapy performs massage while a thermal ceramic is moved in the longitudinal direction along the user's body, and the thermal ceramic is configured to massage the user's body while rotating in the process of repeatedly reciprocating the entire moving section. If the thermal ceramic does not rotate, the fiction between the thermal ceramic and the cover can be maximized and the cover can wear out quickly, and thus, this is to configure the thermal ceramic to rotate naturally due to the friction of the cover.

In the conventional case, a heating element in a non-rotating state which is connected to a power source is inserted into the thermal ceramic in order to heat the rotating heating element, and the thermal ceramics are configured to be spaced apart from each other such that the rotating thermal ceramics can rotate relative to the heating element in a non-rotating state.

However, as the thermal ceramic and the heating element are disposed to be spaced apart from each other, the heat generated from the heating element is not smoothly transferred, and thus, there is a problem in that the thermotherapy effect is reduced.

Therefore, there is a need for improvement in these areas.

### DISCLOSURE

### Technical Tasks

The technical problems to be solved in the present invention are to solve the problems of the related art described above, and are directed to providing a pressure module which is capable of receiving current from a power supply unit in a non-rotating state even in a state where a heat-generating unit for heating a pressure unit rotates together with the pressure unit, and a thermotherapy apparatus including the same.

The problems of the present invention are not limited to the problems that are mentioned above, and other problems that are not mentioned will be clearly understood by those skilled in the art from the following description.

### Technical Solution

In order to solve the above problems, the pressure module according to an exemplary embodiment of the present invention may include a pressure unit which has an interior space; a heat-generating unit which is inserted in the interior space and generates heat such that the pressure unit is heated; and an electromagnetic induction unit which generates an induced current such that the heat-generating unit generates heat.

In this case, the electromagnetic induction unit may be inserted into the heat-generating unit.

In this case, the electromagnetic induction unit may be disposed outside the heat-generating unit.

In this case, the pressure unit and the heat-generating unit may be integrally formed, and the heat-generating unit may be made of a metal material.

In this case, the heat-generating unit may include a first heat-generating unit which is spaced apart from the pressure unit; a second heat-generating unit which extends from one side of the first heat-generating unit, surrounds the first heat-generating unit, and contacts the pressure unit; and a third heat-generating unit which extends from the other side of the first heat-generating unit, surrounds the first heat-generating unit, and contacts the pressure unit, wherein the third heat-generating unit is disposed to be spaced apart from the second heat-generating unit.

In this case, the third heat-generating unit may be disposed symmetrically with the second heat-generating unit.

In this case, the heat-generating unit may include a first heat-generating unit which is spaced apart from the pressure unit; and a plurality of second heat-generating units which extend from the first heat-generating unit to contact the pressure unit, and are formed in a spiral shape that is bent in a direction toward the pressure unit, wherein the plurality of second heat-generating units are disposed to be spaced apart from each other.

In this case, the heat-generating unit may be formed in a spring shape.

In addition, the thermotherapy apparatus according to an exemplary embodiment of the present invention may include a pressure module for thermotherapy; and a driving unit for moving the pressure module, wherein the pressure module may include a pressure unit which has an interior space; a heat-generating unit which is inserted in the interior space and generates heat such that the pressure unit is heated; an electromagnetic induction unit which generates an induced current such that the heat-generating unit generates heat; and a support unit for supporting the pressure module, wherein the heat-generating unit rotates relative to the electromagnetic induction unit in a non-rotating state such that the heat-generating unit rotates together with the pressure module.

In this case, the outer side surface of the heat-generating unit may be in contact with the inner side surface of the pressure unit.

In this case, the electromagnetic induction unit may be inserted into the heat-generating unit, and a separation space may be formed between the heat-generating unit and the electromagnetic induction unit such that heat generated from the heat-generating unit is not directly conducted to the electromagnetic induction unit.

In this case, the thermotherapy apparatus may further include a power supply unit for supplying current to the electromagnetic induction unit, wherein the electromagnetic induction unit is provided with a coil that receives current from the power supply unit to generate an induced current.

In this case, the coil may be formed in a spiral shape that is wound multiple times.

In this case, a heat insulating member may be provided on an outer side surface of the electromagnetic induction unit to prevent heat generated from the heat-generating unit from moving to the coil.

In this case, the heat-generating unit may include a plurality of conductive units that generate heat by generating an induced current by the electromagnetic induction unit, and are disposed to be spaced apart from each other; and a plurality of insulation units that are disposed between the plurality of conductive units.

In this case, the width of the plurality of conductive units may be longer than the width of the plurality of insulation units.

In this case, the thermotherapy apparatus may further include an elevation module for elevating the pressure unit, wherein the electromagnetic induction unit is disposed on the upper side of the elevation module while being disposed outside the pressure unit.

In this case, the thermotherapy apparatus may further include a power supply unit for supplying current to the electromagnetic induction unit, wherein the electromagnetic induction unit is provided with a coil that receives power from the power supply unit to generate an induced current.

### Advantageous Effects

According to an exemplary embodiment of the present invention having the above-described configuration, since the heat-generating unit for heating the pressure unit is configured to rotate together with the pressure unit, the heat generated from the heat-generating unit is smoothly transferred to the pressure unit as the pressure unit and the heat-generating unit are disposed to be in contact with each other, and accordingly, the thermotherapy effect is improved.

In addition, as heat is smoothly transferred from the heat-generating unit to the pressure unit, heat loss is minimized, and the power consumption efficiency of the thermotherapy apparatus is improved.

In addition, since the current is stably supplied even in a state where the heat-generating unit rotating together with the pressure unit rotates relative to the power supply unit in a non-rotating state, it is possible to secure the operation stability of the thermotherapy apparatus.

### DESCRIPTION OF DRAWINGS

FIG. 1 is a cross-sectional view showing the thermotherapy apparatus including a heating module according to an exemplary embodiment of the present invention.

FIG. 2 is a view schematically showing a state in which the heating module is lifted according to an exemplary embodiment of the present invention.

FIG. 3 is a cross-sectional view showing a pressure unit, a heat-generating unit and an electromagnetic induction unit according to an exemplary embodiment of the present invention.

FIG. 4 is a cross-sectional view showing a state in which a pressure unit and a heat-generating unit are separated according to an exemplary embodiment of the present invention.

FIG. 5 is a perspective view showing a state in which a heat-generating unit and an electromagnetic induction unit are separated according to an exemplary embodiment of the present invention.

FIG. 6 is a perspective view showing a heat-generating unit according to another exemplary embodiment of the present invention.

FIG. 7 is a cross-sectional view showing a pressure unit, a heat-generating unit and an electromagnetic induction unit according to still another exemplary embodiment of the present invention.

FIG. 8 is a plan view of a coil according to still another exemplary embodiment of the present invention as viewed from above.

FIG. 9 is a plan view of a coil according to another exemplary embodiment of the present invention as viewed from above.

FIG. 10 is a flowchart showing a method of coupling the heat-generating unit and the electromagnetic induction unit to the pressure unit according to an exemplary embodiment of the present invention.

FIG. 11 is a cross-sectional view showing a heat-generating unit according to still another exemplary embodiment of the present invention.

FIG. 12 is a cross-sectional view showing a heat-generating unit according to still another exemplary embodiment of the present invention.

FIG. 13 is a cross-sectional view showing a heat-generating unit according to still another exemplary embodiment of the present invention.

| | |
|---|---|
| 1: Thermotherapy apparatus | 10: Pressure module |
| 100: Pressure unit | 200: Heat-generating unit |
| 300: Electromagnetic induction unit | 500: Support unit |

### MODES OF THE INVENTION

Hereinafter, various exemplary embodiments will be described more specifically with reference to the accompanying drawings. The exemplary embodiments may be variously modified. Specific exemplary embodiments may be depicted in the drawings and concretely explained in the detailed description. However, specific exemplary embodiments disclosed in the accompanying drawings are only intended to facilitate understanding of various exemplary embodiments. Therefore, it is not intended to limit the technical idea to the specific exemplary embodiments disclosed in the accompanying drawings, and it should be understood to include all equivalents or substitutes included in the spirit and scope of the invention.

Terms such as first or second may be used to describe various components, but the components should not be limited by the terms. The terms are only used for the purpose of distinguishing one component from another component.

In an exemplary embodiment of the present invention, it should be understood that the term “include” or “have” indicates that a feature, a number, a step, an operation, a component, a part or the combination thereof described in an exemplary embodiment of the present invention is present, but does not exclude the possibility of presence or addition of one or more other features, numbers, steps, operations, components, parts or combinations thereof, in advance. When a component is said to be “connected” or “joined” to another component, it may be directly connected or joined to that other component, but it is to be understood that other components may exist in between. On the other hand, when a component is said to be “directly connected” or “directly joined” to another component, it should be understood that there is no other component in between.

Meanwhile, “a module” or “a unit, part or portion” for a component used in an exemplary embodiment of the present invention performs at least one function or operation. In addition, the “module” or “unit, part or portion” may perform a function or operation by hardware, software or a combination of hardware and software. In addition, a plurality of “modules” or a plurality of “units, parts or portions” except for modules” or “units, parts or portions” that should be performed in a specific hardware or is performed by at least one processor may be integrated into at least one module. Singular expressions used herein include plural expressions unless they have definitely opposite meanings in the context.

Further, in terms of describing the present invention, when it is determined that the specific description about the related known technique may unnecessarily obscure the gist of the present invention, the detailed description thereof is abbreviated or omitted.

In an exemplary embodiment of the present invention, the direction of the arrow on the Z-axis is referred to as the upper side of the thermotherapy apparatus, and the lower side is referred to as the opposite direction to the upper side.

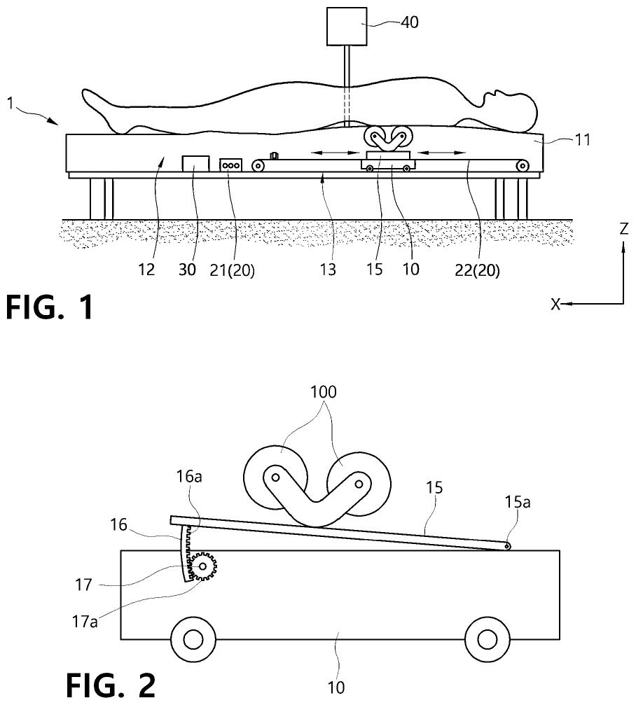

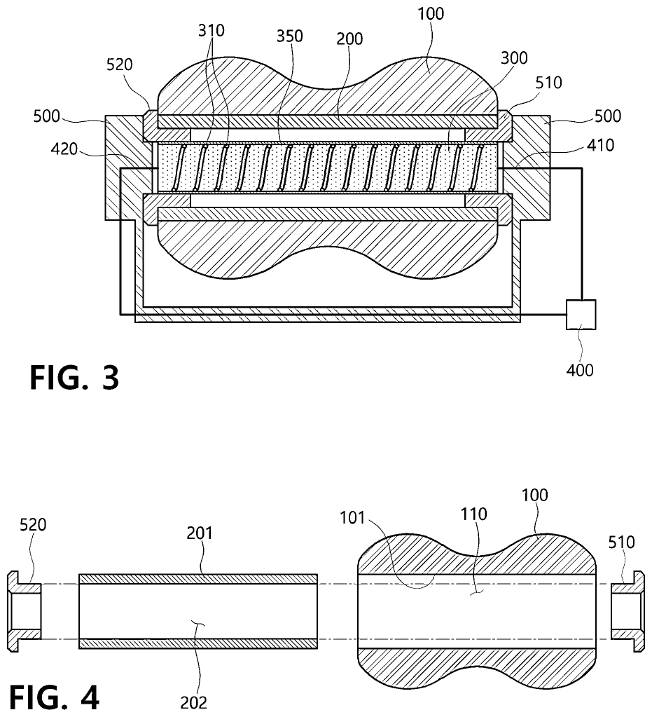

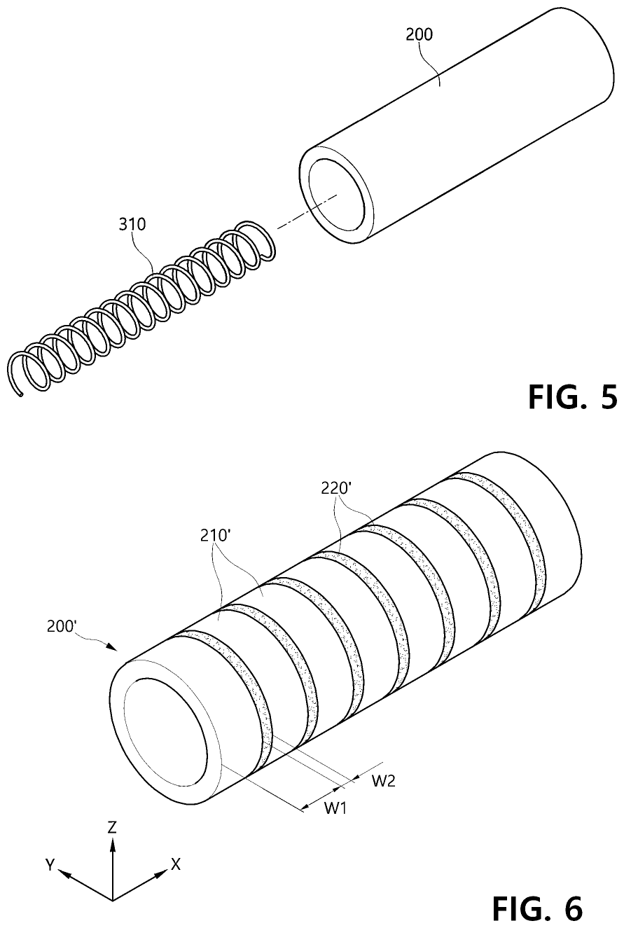

FIG. 1 is a cross-sectional view showing the thermotherapy apparatus according to an exemplary embodiment of the present invention, FIG. 2 is a view schematically showing a state in which the heating module is lifted according to an exemplary embodiment of the present invention, FIG. 3 is a cross-sectional view showing a pressure unit, a heat-generating unit and an electromagnetic induction unit according to an exemplary embodiment of the present invention, FIG. 4 is a cross-sectional view showing a state in which a pressure unit and a heat-generating unit are separated according to an exemplary embodiment of the present invention, and FIG. 5 is a perspective view showing a state in which a heat-generating unit and an electromagnetic induction unit are separated according to an exemplary embodiment of the present invention.

As illustrated in FIGS. 1 and 2, the thermotherapy apparatus 1 according to an exemplary embodiment of the present invention includes a pressure module 10, a driving unit 20 for moving the pressure module 10, a control unit 30 for controlling the operation of the driving unit 20 and an input unit 40 for inputting a desired thermotherapy pattern by the user.

In this case, the thermotherapy apparatus 1 includes a main mat 11 which is used for the user's upper body and his spine part, and an auxiliary mat 12 which is used for the user's lower body. In addition, the thermotherapy apparatus 1 may include a mounting unit 13 for placing and supporting the main mat 11 and the auxiliary mat 12 as needed.

The pressure module 10 massages the spine while moving in the longitudinal direction (X-axis) along the user's spine. In addition, the pressure module 10 provides a warm compress and massage effect to the user by using the high-temperature heat that is generated by using current supplied from a power supply unit, which will be described below.

In this case, the pressure module 10 may be configured to provide a warm compress and massage effect to the user by using not only high-temperature heat but also far-infrared rays.

The pressure unit 100 provided in the pressure module 10 is formed in a roller shape. However, the pressure unit 100 is not limited to being formed in a roller shape, and various shapes and structures are possible as long as the pressure unit 100 is rotated while the pressure module 10 is moved.

In addition, as illustrated in FIG. 2, the pressure module 10 is provided with an elevation module 15 for elevating the pressure unit 100.

One side 15a of the elevation module 15 is hinge-coupled to the pressure module 10. In addition, the other side of the elevation module 15 is coupled to a rack 16 which is formed with a ack gear 16a. In this case, the ack 16 is formed to protrude downward of the elevation module 15. In addition, the pressure module 10 is provided with a pinion 17 in which a pinion gear 17a meshing with the rack gear 16a is formed. As the pinion gear 17 rotates, the rack 16 moves upward or downward. As the rack 16 moves in an upward or downward direction, the pressure unit 100 in the elevation module 15 is raised or lowered.

Accordingly, in the elevation module 15, the pressure unit 100 presses the user's spine with a certain force in response to the protruding shape of the user's spine. For example, when the pressure unit 100 presses the user's spine with a force that is greater than a certain force, the elevation module 15 moves the pressure unit 100 downward. Conversely, when the pressure unit 100 presses the user's spine with a force that is smaller than a certain force, the elevation module 15 moves the pressure unit 100 upward. That is, the elevation module 15 elevates the pressure unit 100 in response to the protruding shape of the user's spine.

The driving unit 20 moves the pressure module 10 in the longitudinal direction (X-axis) of the user's spine. The driving unit 20 includes a driving motor 21 for providing a driving force to reciprocate the pressure module 10, and a transfer member 22 for receiving a driving force from the driving motor 21 to move the pressure module 10.

The driving motor 21 receives current to generate a driving force. In addition, the transfer member 22 is connected to the driving motor 21 to move the pressure module 10 according to the rotation of the driving motor 21.

In this case, the transfer member 22 may be selectively implemented among a conveying belt, a conveying chain and a conveying rope, and in addition, similar to a method of using a rack and a pinion, various structures and methods that are capable of transferring the pressing module 10 by receiving a driving force of the driving motor 21 are possible.

In addition, the driving motor 21 may be disposed to be spaced apart from the pressure module 10 to provide a driving force to the pressure module 10, or may be mounted inside the pressure module 10.

The control unit 30 controls the operation of the driving unit 20 according to a user input value or a preset value. In addition, the control unit 30 is electrically connected to the elevation module 15 to control the force by which the pressure unit 100 presses the user's body by the elevation module 15.

In addition, according to various exemplary embodiments of the present invention, the control unit 30 may include a communication module (not illustrated) for transmitting and receiving electrical signals to and from an input unit 40 to be described below by wire or wirelessly. The control unit 30 having the communication module may receive an electrical signal to control the operation of the driving unit 20.

The input unit 40 generates an electrical signal for providing a desired thermotherapy pattern by a user's manipulation. In addition, the input unit 40 is electrically connected to the control unit 30 to transmit the electrical signal to the control unit 30.

In addition, according to various exemplary embodiments of the present invention, the input unit 40 may be an electronic device such as a smart phone, and the thermotherapy apparatus 1 may be provided with a cradle for mounting an electronic device such as a smart phone.

As illustrated in FIG. 3, the pressure module 10 includes a pressure unit 100 having an interior space 110 formed therein, a heat-generating unit 200 which is inserted into the interior space 110, an electromagnetic induction unit 300 for generating an induced current such that the heat-generating unit 200 generates heat, a power supply unit 400 for supplying current to the electromagnetic induction unit 300, and a support unit 500 for supporting the pressure unit 100.

As illustrated in FIG. 5, the heat-generating unit 200 has an approximately cylindrical shape. The heat-generating unit 200 is made of a metal material. However, the heat-generating unit 200 is not limited to a metal material, and it may be made of various materials having good conductivity and good thermal conductivity, such as graphite, conductive plastic or conductive silicon.

The heat-generating unit 200 is integrally formed with the pressure unit 100. In addition, the outer side surface 201 of the heat-generating unit 200 is in surface contact with the inner side surface 101 of the pressure unit 100. In this case, in the thermotherapy process, the heat-generating unit 200 is configured to rotate together with the pressure unit 100. Accordingly, the heat generated from the heat-generating unit 200 is smoothly transferred to the pressure unit 100, thereby improving the efficiency of power energy for the heat-generating unit 200 to generate heat.

Meanwhile, the entire outer side surface of the heat-generating unit 200 may contact the entire inner side surface of the pressure unit 100, but the present invention is not necessarily limited thereto, and as long as heat generated from the heat-generating unit 200 may be smoothly transferred to the pressure unit 100, it is also possible to be configured to partially contact each other.

In this way, as heat is smoothly transferred from the heat-generating unit 200 to the pressure unit 100, heat loss is minimized, and the power consumption efficiency of the thermotherapy apparatus is improved.

The electromagnetic induction unit 300 is inserted into an interior 202 of the heat-generating unit 200. The outer appearance of the electromagnetic induction unit 300 is approximately formed in a cylindrical shape. The electromagnetic induction unit 300 is provided with a coil 310 that receives a current to generate an induced current.

In this case, the coil 310 is formed a spiral shape that is wound multiple times. However, the coil 310 is not limited to being formed in a spiral shape, and it may be formed in various shapes and structures for generating an induced current.

The coil 310 generates an induced current, and the induced current generated by the coil 310 generates a current in the heat-generating unit 200. In this case, the current flowing through the heat-generating unit 200 is directly converted to heat energy, and the heat-generating unit 200 generates heat.

In addition, the electromagnetic induction unit 300 is fixed in a non-rotating state. The heat-generating unit 200 generates an induced current by the electromagnetic induction unit 300 while rotating relative to the electromagnetic induction unit 300, so as not to interfere with the electromagnetic induction unit 300 to enable the stable operation of the thermotherapy apparatus 1.

In addition, a separation space 210 is formed between the heat-generating unit 200 and the electromagnetic induction unit 300 such that heat generated from the heat-generating unit 200 is not directly conducted to the electromagnetic induction unit 300. That is, the separation space 210 reduces the reverse inflow of the heat generated from the heat-generating unit 200 into the electromagnetic induction unit 300, and thus, the heat of the heat-generating unit 200 is efficiently transferred to the pressure unit 100.

In addition, the electromagnetic induction unit 300 is disposed in the center of the inside of the heat-generating unit 200. Accordingly, since the electromagnetic induction unit 300 is disposed to be spaced apart from the heat-generating unit 200 at the same interval and the heat-generating unit 200 heats uniformly as a whole, it is possible to prevent the temperature deviation of the heat-generating unit 200 from occurring.

In addition, according to an exemplary embodiment of the present invention, as the heat-generating unit 200 made of a metal material is provided in a shape to surround the electromagnetic induction unit 300, the heat-generating unit 200 which is made of a metal material blocks electromagnetic waves generated by the electromagnetic induction unit 300 from flowing out to the outside through the pressure unit 100.

In addition, a heat insulating member 350 for preventing the heat generated from the heat-generating unit 200 from moving to the coil 310 may be provided on the outer side surface of the electromagnetic induction unit 300.

The heat insulating member 350 is formed to surround the outer side surface of the electromagnetic induction unit 300. In addition, the heat insulating member 350 is made of a polymer resin having heat resistance. In addition, the heat insulating member 350 is not limited to a heat-resistant polymer resin, and it may be made of various materials having heat resistance, such as glass or quartz. In this way, the heat insulating member 350 prevents the heat generated from the heat-generating unit 200 from flowing back into the electromagnetic induction unit 300, and thus, the heat of the heat-generating unit 200 is efficiently transferred to the pressure unit 100.

The power supply unit 400 is electrically connected to the coil 310. The power supply unit 400 supplies current to the coil 310. The power supply unit 400 includes a first electrode 410 which is connected to one end of the coil 310 and a second electrode 420 which is connected to the other end of the coil 310. Accordingly, the first electrode 410 and the second electrode 420 electrically connect the power supply unit 400 and the coil 310.

As illustrated in FIG. 3, the support unit 500 supports the pressure unit 100 to be rotatable. In this case, the support unit 500 is provided with a first bushing 510 which is provided on one side of the pressure unit 100 and a second bushing 520 which is provided on the other side of the pressure unit 100.

The second bushing 520 is provided to face the first bushing 510 with the heat-generating unit 200 interposed therebetween.

In a state where the first bushing 510 is coupled to one side of the pressure unit 100, the heat-generating unit 200 is inserted into the interior space 110 of the pressure unit 100. In addition, after the electromagnetic induction unit 300 is inserted into the interior 202 of the heat-generating unit 200, the second bushing 520 is coupled to the other side of the pressure unit 100.

The first bushing 510 and the second bushing 520 are provided such that the pressure unit 100 and the heat generating part 200 are rotatably supported by the support unit 500.

FIG. 6 is a perspective view showing a heat-generating unit according to another exemplary embodiment of the present invention.

As illustrated in FIG. 6, according to another exemplary embodiment of the present invention, the heat-generating unit 200′ includes a plurality of conductive units 210′ for generate heat by generating an induced current by the electromagnetic induction unit 300 (refer to FIG. 3), and a plurality of insulation units 220′ that are disposed between the plurality of conductive units 210′. In addition, the above description will be substituted for the same or similar components as those of the above-described exemplary embodiment.

The plurality of conductive units 210′ are disposed to be spaced apart from each other. The plurality of conductive units 210′ are formed in a ring shape. However, the plurality of conductive units 210′ are not limited to being formed in a ring shape, and they may be formed in various shapes forming a closed loop.

In addition, the width W1 of the plurality of conductive units 210′ may be constant. In addition, according to various exemplary embodiments of the present invention, the width W1 of the plurality of conductive units 210′ may decrease toward the center of the heat-generating unit 200′.

The plurality of insulation units 220′ electrically insulate the plurality of conductive units 210′. The plurality of insulation units 220′ are formed in a ring shape when the plurality of conductive units 210′ are formed in a ring shape.

The plurality of insulation units 220′ are made of quartz or glass. However, the plurality of insulation units 220′ are not limited to those made of quartz or glass, and they may be made of various materials that block the flow of electricity.

As the plurality of insulation units 220′ electrically insulate the plurality of conductive units 210′, the plurality of conductive units 210′ individually generate an induced current. That is, the plurality of conductive units 210′ individually generate an induced current by the electromagnetic induction unit 300 (refer to FIG. 3), and thus, the heating performance of the heat-generating unit 300′ may be improved.

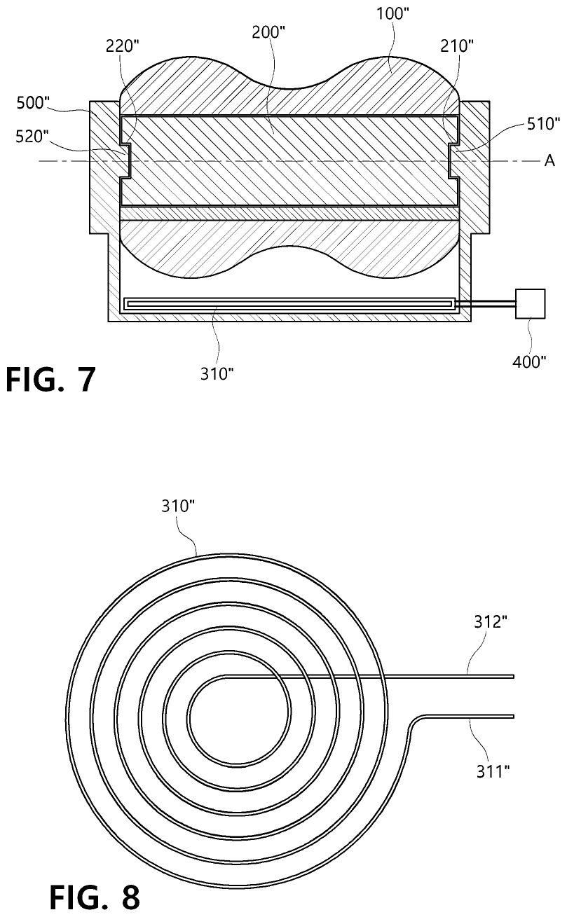

FIG. 7 is a cross-sectional view showing a pressure unit, a heat-generating unit and an electromagnetic induction unit according to still another exemplary embodiment of the present invention, and FIG. 8 is a plan view of a coil according to still another exemplary embodiment of the present invention as viewed from above.

Referring to FIGS. 7 and 8, the thermotherapy apparatus 1′ according to still another exemplary embodiment of the present invention includes a pressure unit 100″, a heat-generating unit 200″, an electromagnetic induction unit 300″, a power supply unit 400″ and a support unit 500″. In addition, the same or similar components to the components of the above-described exemplary embodiment will be replaced with the above description, and the electromagnetic induction unit 300″ will be mainly described.

First of all, the heat-generating unit 200″ is filled in the interior space of the pressure unit 100″. Accordingly, the heat-generating unit 200″ rotates stably together with the pressure unit 100″.

A first support groove 210″ is formed on one side of the heat-generating unit 200″, and a second support groove 220″ which is disposed to face the first support groove 210″ is formed on the other side of the heat-generating unit 200″.

In addition, the support unit 500″ is provided with a first support protrusion 510″ which is inserted into the first support groove 210″ and a second support protrusion 520″ which is inserted into the second support groove 220″.

The first support protrusion 510″ and the second support protrusion 520″ are formed on the rotation shaft A of the heat generating part 200″. The first support protrusion 510″ and the second support protrusion 520″ stably support the heat-generating unit 200″ to rotate. Accordingly, the pressure unit 100′ is also rotated while being supported by the support unit 500″ together with the heat-generating unit 200″.

The electromagnetic induction unit 300″ is disposed outside the pressure unit 100″. In addition, the electromagnetic induction unit 300″ is disposed above the elevation module 15 (refer to FIG. 1).

As illustrated in FIG. 8, the electromagnetic induction unit 300″ is provided with a coil 310″ having a flat plate shape which is wound multiple times.

The coil 310″ has a circular shape as a whole when viewed from above. One end 311″ of the coil 310″ and the other end 312″ of the coil 310″ are electrically connected to the power supply unit 400″.

Accordingly, the coil 310″ receives current from the power supply unit 400″ to generate an induced current in the heating element 200″. In addition, the heat-generating unit 200″ is heated by the induced current, and the heat generated from the heat-generating unit 200″ is transferred to the pressure unit 100″.

As such, according to still another exemplary embodiment of the present invention, even when the electromagnetic induction unit 300″ is disposed outside the pressure unit 100″, by heating the heat-generating unit 200″, the pressure unit 100″ is rotated stably together with the heat-generating unit 200″.

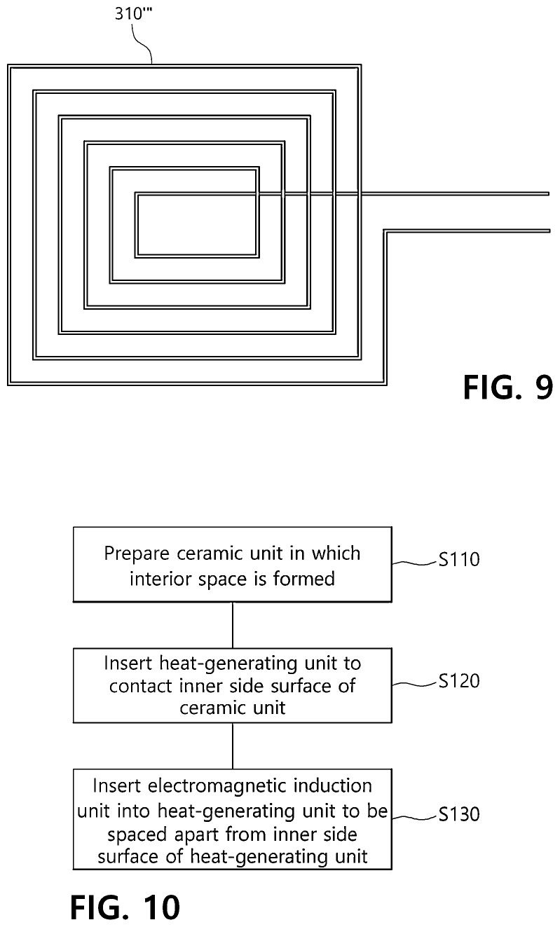

FIG. 9 is a plan view of a coil according to another exemplary embodiment of the present invention as viewed from above.

As illustrated in FIG. 9, the coil 310′″ according to still another exemplary embodiment of the present invention has a substantially rectangular shape when viewed from above, unlike the coil of the above-described exemplary embodiment.

That is, the coil 310′″ having a rectangular shape is provided to correspond to the lower side of the heat-generating unit 200″ (refer to FIG. 7). For example, the lower side of the heat-generating unit 200″ (refer to FIG. 7) may have a substantially rectangular shape when viewed from the lower side. Accordingly, since the heat-generating unit 200″ (refer to FIG. 7) generates an induced current by the coil 310′″ having a rectangular shape, the heat-generating performance of the heat-generating unit 200″ (refer to FIG. 7) may be improved.

FIG. 10 is a flowchart showing a method of coupling the heat-generating unit and the electromagnetic induction unit to the pressure unit according to an exemplary embodiment of the present invention.

As illustrated in FIG. 10, the method of coupling the heat-generating unit and the electromagnetic induction unit to the pressure unit according to an exemplary embodiment of the present invention includes the steps of preparing a pressure unit S110, inserting a heat-generating unit S120, and inserting an electromagnetic induction unit S130.

In the step of preparing the pressure unit S110, the pressure unit having an interior space is prepared. Herein, the interior space is formed in a cylindrical shape.

In the step of inserting the heat-generating unit S120, the heat-generating unit having an outer side surface corresponding to the inner side surface of the pressure unit is first prepared. The heat-generating unit has a cylindrical shape. The heat-generating unit is inserted into the interior space of the pressure unit. In this case, the outer side surface of the heat-generating unit is in contact with the inner side surface of the pressure unit. In addition, an adhesive is applied to the outer side surface of the heat-generating unit, and the outer side surface of the heat-generating unit is coupled to the inner side surface of the pressure unit by an adhesive.

In the step of inserting the electromagnetic induction unit S130, the electromagnetic induction unit to be inserted into the heat-generating unit is prepared. The electromagnetic induction unit also has a cylindrical shape. An outer diameter of the electromagnetic induction unit is smaller than an outer diameter of the heat-generating unit. That is, the electromagnetic induction unit is inserted into the heat-generating unit to be spaced apart from the inner side surface of the heat-generating unit.

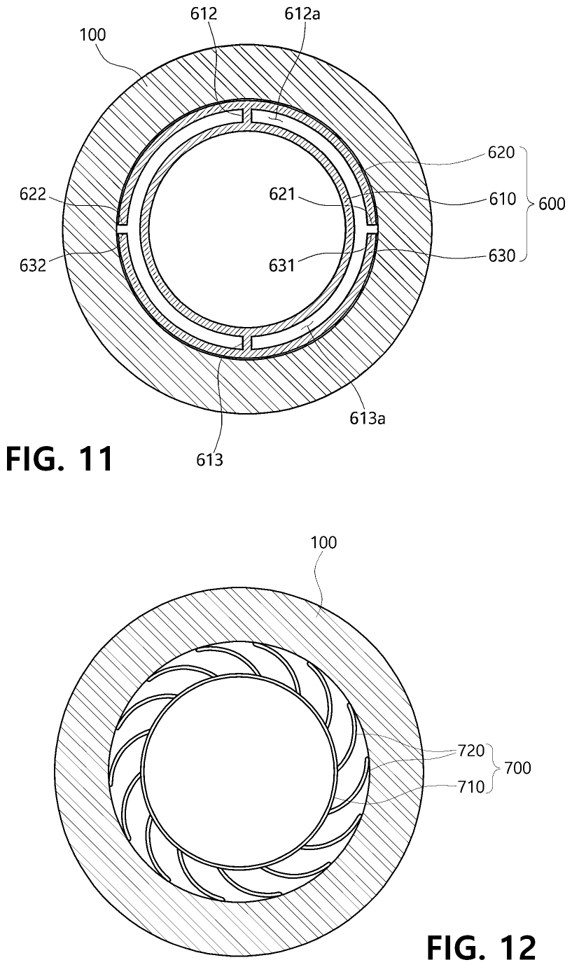

FIG. 11 is a cross-sectional view showing a heat-generating unit according to still another exemplary embodiment of the present invention.

As illustrated in FIG. 11, the heat-generating unit 600 according to still another exemplary embodiment of the present invention includes a first heat-generating unit 610, a second heat-generating unit 620 and a third heat-generating unit 630, unlike the above-described exemplary embodiment.

The first heat-generating unit 610 is disposed to be spaced apart from the pressure unit 100. The first heat-generating unit 610 may have a cylindrical shape. Accordingly, since the first heat-generating unit 610 is disposed to be spaced apart from the pressure unit 100, the pressure unit 100 is prevented from being damaged by thermal expansion of the first heat-generating unit 610.

The second heat-generating unit 620 is provided with a first extension unit 612 which extends from one side of the first heat-generating unit 610. Accordingly, the second heat-generating unit 620 is connected to the first heat-generating unit 610 by the first extension unit 612. In addition, a first separation space 612a is provided to be formed between the second heat-generating unit 620 and the first heat-generating unit 610 to surround the first heat-generating unit 610, so as to be in contact with the pressure unit 100.

The third heat-generating unit 630 is provided with a second extension unit 613 which extends from the other side of the first heat-generating unit 610. Accordingly, the third heat-generating unit 630 is connected to the first heat-generating unit 610 by the second extension unit 613. In addition, a second separation space 613a is provided to be formed between the third heat-generating unit 630 and the first heat-generating unit 610 to surround the first heat-generating unit 610, so as to be in contact with the pressure unit 100. In this case, the third heat-generating unit 630 is disposed to be spaced apart from the second heat-generating unit 620. For example, one end 621 of the second heat-generating unit 620 and one end 631 of the third heat-generating unit 630 are spaced apart from each other, and the other end 622 of the second heat-generating unit 620 and the other end 632 of the third heat-generating unit 633 are spaced apart from each other.

In addition, the third heat-generating unit 630 is formed symmetrically with the second heat-generating unit 620 with the first heat-generating unit 610 interposed therebetween.

In addition, as the second heat-generating unit 620 and the third heat-generating unit 630 come into contact with the pressure unit 100, the heat-generating unit 610 is stably disposed inside the pressure unit 100 while being in contact with the pressure unit 100.

As the heat-generating unit 600 generates heat, even if the second heat-generating unit 620 and the third heat-generating unit 630 are thermally expanded, the first heat-generating unit 610, the second heat-generating unit 620 and the third heat-generating unit 630 are disposed to be spaced apart from each other, and thus, the pressure unit 100 is prevented from being damaged due to thermal expansion of the second heat-generating unit 620 and the third heat-generating unit 630.

FIG. 12 is a cross-sectional view showing a heat-generating unit according to still another exemplary embodiment of the present invention.

As illustrated in FIG. 12, the heat-generating unit 700 according to still another exemplary embodiment of the present invention includes a first heat-generating unit 710 and a plurality of second heat-generating units 720, unlike the above-described exemplary embodiment.

The first heat-generating unit 710 is disposed to be spaced apart from the pressure unit 100. The first heat-generating unit 710 may have a cylindrical shape. Accordingly, since the first heat-generating unit 710 is disposed to be spaced apart from the pressure unit 100, the pressure unit 100 is prevented from being damaged by thermal expansion of the first heat-generating unit 710.

The plurality of second heat-generating units 720 extend from the first heat-generating units 710. The plurality of second heat-generating units 720 are in contact with the pressure unit 100. The plurality of second heat-generating units 720 have a spiral shape that is bent in a direction toward the pressure unit 100. For example, the plurality of second heat-generating units 720 have a spiral shape that is bent in a direction of surrounding the first heat-generating unit 710.

In this case, the plurality of second heat-generating units 720 are disposed to be spaced apart from each other. Accordingly, as the heat-generating unit 700 generates heat, even if the plurality of second heat-generating units 720 are thermally expanded, the plurality of second heat-generating units 720 are disposed to be spaced apart from each other, and thus, the pressure unit 100 is prevented from being damaged by thermal expansion of the plurality of second heat-generating units 720.

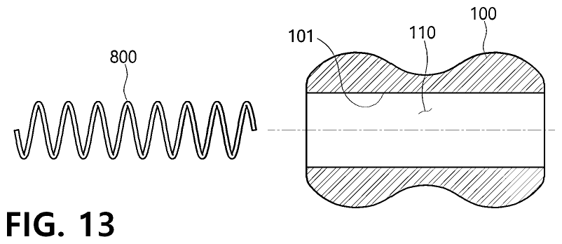

FIG. 13 is a cross-sectional view showing a heat-generating unit according to still another exemplary embodiment of the present invention.

As illustrated in FIG. 13, the heat-generating unit 800 according to still another exemplary embodiment of the present invention is formed in a spring shape, unlike the above-described exemplary embodiment.

Even if the heat-generating unit 800 is thermally expanded, since it thermally expands in the longitudinal direction due to a spring shape rather than in the direction in which the pressure unit 100 is pressed, the pressure unit 100 is prevented from being damaged by thermal expansion of the heat-generating unit 800.

As described above, the preferred exemplary embodiments according to the present invention have been described, and in addition to the above-described exemplary embodiments, the fact that the present invention can be embodied in other specific forms without departing from the spirit or scope of the present invention will be apparent to those having ordinary skill in the art. Therefore, the above-described exemplary embodiments are to be regarded as illustrative rather than restrictive, and accordingly, the present invention is not limited to the above description, but may be modified within the scope of the appended claims and their equivalents.

1. A pressure module, comprising:
a pressure unit which has an interior space;
a heat-generating unit which is inserted in the interior space and generates heat such that the pressure unit is heated; and
an electromagnetic induction unit which generates an induced current such that the heat-generating unit generates heat.

2. The pressure module of claim 1, wherein the electromagnetic induction unit is inserted into the heat-generating unit.

3. The pressure module of claim 1, wherein the electromagnetic induction unit is disposed outside the heat-generating unit.

4. The pressure module of claim 1, wherein the pressure unit and the heat-generating unit are integrally formed, and the heat-generating unit is made of a metal material.

5. The pressure module of claim 1, wherein the heat-generating unit comprises:
a first heat-generating unit which is spaced apart from the pressure unit;
a second heat-generating unit which extends from one side of the first heat-generating unit, surrounds the first heat-generating unit, and contacts the pressure unit; and
a third heat-generating unit which extends from the other side of the first heat-generating unit, surrounds the first heat-generating unit, and contacts the pressure unit,
wherein the third heat-generating unit is disposed to be spaced apart from the second heat-generating unit.

6. The pressure module of claim 5, wherein the third heat-generating unit is disposed symmetrically with the second heat-generating unit.

7. The pressure module of claim 1, wherein the heat-generating unit comprises:
a first heat-generating unit which is spaced apart from the pressure unit; and
a plurality of second heat-generating units which extend from the first heat-generating unit to contact the pressure unit, and are formed in a spiral shape that is bent in a direction toward the pressure unit,
wherein the plurality of second heat-generating units are disposed to be spaced apart from each other.

8. The pressure module of claim 1, wherein the heat-generating unit is formed in a spring shape.

9. A thermotherapy apparatus, comprising:
a pressure module for thermotherapy; and
a driving unit for moving the pressure module,
wherein the pressure module comprises:
a pressure unit which has an interior space;
a heat-generating unit which is inserted in the interior space and generates heat such that the pressure unit is heated;
an electromagnetic induction unit which generates an induced current such that the heat-generating unit generates heat; and
a support unit for supporting the pressure module,
wherein the heat-generating unit rotates relative to the electromagnetic induction unit in a non-rotating state such that the heat-generating unit rotates together with the pressure module.

10. The thermotherapy apparatus of claim 9, wherein the outer side surface of the heat-generating unit is in contact with the inner side surface of the pressure unit.

11. The thermotherapy apparatus of claim 10, wherein the electromagnetic induction unit is inserted into the heat-generating unit, and
wherein a separation space is formed between the heat-generating unit and the electromagnetic induction unit such that heat generated from the heat-generating unit is not directly conducted to the electromagnetic induction unit.

12. The thermotherapy apparatus of claim 11, further comprising:
a power supply unit for supplying current to the electromagnetic induction unit,
wherein the electromagnetic induction unit is provided with a coil that receives current from the power supply unit to generate an induced current.

13. The thermotherapy apparatus of claim 12, wherein the coil is formed in a spiral shape that is wound multiple times.

14. The thermotherapy apparatus of claim 12, wherein a heat insulating member is provided on an outer side surface of the electromagnetic induction unit to prevent heat generated from the heat-generating unit from moving to the coil.

15. The thermotherapy apparatus of claim 9, wherein the heat-generating unit comprises:
a plurality of conductive units that generate heat by generating an induced current by the electromagnetic induction unit, and are disposed to be spaced apart from each other; and
a plurality of insulation units that are disposed between the plurality of conductive units.

16. The thermotherapy apparatus of claim 15, wherein the width of the plurality of conductive units is longer than the width of the plurality of insulation units.

17. The thermotherapy apparatus of claim 16, further comprising:
an elevation module for elevating the pressure unit,
wherein the electromagnetic induction unit is disposed on the upper side of the elevation module while being disposed outside the pressure unit.

18. The thermotherapy apparatus of claim 16, further comprising:
a power supply unit for supplying current to the electromagnetic induction unit,
wherein the electromagnetic induction unit is provided with a coil that receives power from the power supply unit to generate an induced current.
